(19) United States
(12) Patent Application Publication
(10) Pub. No.: US 2004/0132720 A1
(43) Pub. Date: Jul. 8, 2004

(54) Piperazine derivatives, their preparation and uses in therapy (5ht1b receptor activity)

(76) Inventors: Howard Marshall, Essex (GB); Mervyn Thompson, Essex (GB); Paul Adrian Wyman, Essex (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/471,432

(22) PCT Filed: Mar. 11, 2002
(86) PCT No.: PCT/EP02/02634

(30) Foreign Application Priority Data
Mar. 16, 2001 (GB) 0106586.1

(51) Int. Cl. A61K 31/55; A61K 31/496; C 07D 4 3/14

(52) U.S. Cl. 514/217.05; 514/254.09; 544/360; 544/373; 540/598

(57) ABSTRACT

Compounds of formula (I) or a pharmaceutically acceptable salt thereof are disclosed: in which R^{a} is a group of formula (i) wherein P¹ is phenyl, naphthyl or heteroaryl; R¹ is halogen, C₁₋₆alkyl, C₃₋₆cycloalkyl, COC₁₋₆alkyl, C₁₋₆alkoxy, hydroxy, hydroxyC₁₋₆alkyl, nitro, haloC₁₋₆alkyl, cyano, SR⁶, SOR⁶, SO₂R⁶, SO₂NR⁶R⁷, CO₂R⁶, CONR⁶R⁷, OCONR⁶COR⁷, NR⁶r⁷, NR⁶COR⁷, NR⁶CO₂R⁷, NR⁶SO₂R⁷, NR⁶CONR⁷R⁸, CH₂NR⁶COR⁷, CH₂NR⁶CO₂R⁷, CH₂NR⁶SO₂R⁷, CR⁶═NOR⁷ where R⁶, R⁷ and R⁸ are independently hydrogen or C₁₋₆alkyl, a is 1, 1, 2, or 3; or R^{a} is a group of formula (ii) where P² is phenyl, naphthyl, heteroaryl or a 5 to 7 membered heterocyclic ring; P³ is phenyl, naphthyl or heteroaryl; R² is a defined above for R¹ in formula (I) or R² is heteroaryl optionally substituted By C₁alkyl, halogen or COC₁₋₆alkyl or is a 5-7 membered heterocyclic ring optionally substituted by oxo; R³ is halogen, C₁₋₆alkyl, C₃₋₆cycloalkyl, C₁₋₆alkyl, COC₁₋₆alkyl, hydroxy, intro, haloC₁₋₆alkyl, cyano, CO₂R⁶, CONR⁶R⁷, NR⁶R⁷ where R⁶ and R⁷ are as defined above; b and c are independently 0, 1, 2, or 3; Y is a single bond, CH₂ or NH; X is oxygen, sulfur or N—R⁵ where R⁵ is hydrogen or C₁₋₆alkyl; R^{b} is hydrogen, halogen, C₁₋₆alkyl, haloC₁₋₆alkyl, COC₁₋₆alkyl or cyano; R^{c} is hydrogen or C₁₋₆alkyl. Processes for their preparation, pharmaceutical compositions containing them and their use in therapy as 5-HT_{1B} receptor antagonists, for diseases such as depression, are also disclosed.

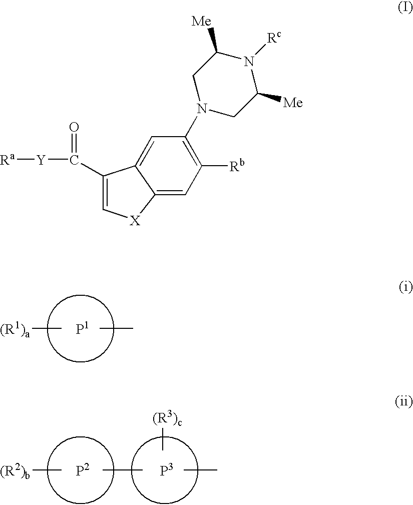

The present invention relates to novel piperazine derivatives, processes for their preparation, pharmaceutical compositions containing the same and to their use in the treatment of CNS and other disorders.

WO 95/06637 discloses a series of piperazine derivatives which are said to possess 5-HT_{1D} receptor antagonist activity. These compounds are alleged to be of use in the treatment of various CNS disorders such as depression. The human 5-HT_{1D} receptor is now known to be encoded by two distinct genes initially designated 5-HT_{1Dα} and 5-HT_{1Dβ} and subsequently redesignated as 5-HT_{1D} and 5-HT_{1B} respectively (P. R. Hartig et al, Trends in Pharmacological Science, 1996, 17, 103-105). WO 98/50538 and WO 98/47885 disclose a series of piperazine derivatives that are said to exhibit combined 5-HT_{1A}, 5-HT_{1B} and 5-HT_{1D} receptor antagonist activity.

A structurally novel class of compounds has now been found which exhibit 5-HT_{1B} receptor activity. In a first aspect, the present invention therefore provides a compound of formula (I) or a pharmaceutically acceptable salt thereof:

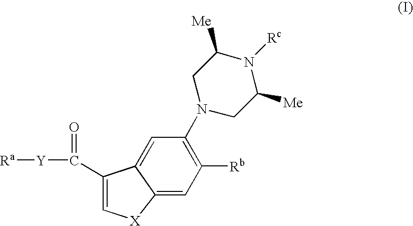

in which R^{a} is a group of formula (i)

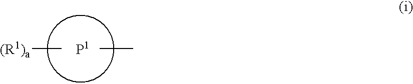

wherein P¹ is phenyl, naphthyl or heteroaryl;

R¹ is halogen, C₁₋₆alkyl, C₃₋₆cycloalkyl, COC₁₋₆alkyl, C₁₋₆alkoxy, hydroxy, hydroxyC₁₋₆alkyl, nitro, haloC₁₋₆alkyl, cyano, SR⁶, SOR⁶, SO₂R⁶, SO₂NR⁶R⁷, CO₂R⁶, CONR⁶R⁷, OCONR⁶R⁷, NR⁶R⁷, NR⁶COR⁷, NR⁶CO₂R⁷, NR⁶SO₂R⁷, NR⁶CONR⁷R⁸, CH₂NR⁶COR⁷, CH₂NR⁶CO₂R⁷, CH₂NR⁶SO₂R⁷, CR⁶═NOR⁷ where R⁶, R⁷ and R⁸ are independently hydrogen or C₁₋₆alkyl,

a is 0, 1, 2 or 3;

or R^{a} is a group of formula (ii)

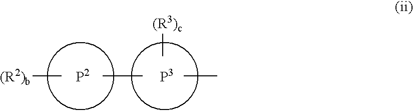

wherein

P² is phenyl, naphthyl, heteroaryl or a 5 to 7 membered heterocyclic ring;

P³ is phenyl, naphthyl or heteroaryl;

R² is as defined above for R¹ in formula (i) or R² is heteroaryl optionally substituted by C₁₋₆alkyl, halogen or COC₁₋₆alkyl or is a 5-7 membered heterocyclic ring optionally substituted by oxo;

R³ is halogen, C₁₋₆alkyl, C₃₋₆cycloalkyl, C₁₋₆alkoxy, COC₁₋₆alkyl, hydroxy, nitro, haloC₁₋₆alkyl, cyano, CO₂R⁶, CONR⁶R⁷, NR⁶R⁷ where R⁶ and R⁷ are as defined above;

b and c are independently 0, 1, 2 or 3;

Y is a single bond, CH₂ or NH;

X is oxygen, sulfur or N—R⁵ where R⁵ is hydrogen or C₁₋₆alkyl;

R^{b} is hydrogen, halogen, C₁₋₆alkyl, haloC₁₋₆alkyl, COC₁₋₆alkyl or cyano;

R^{c} is hydrogen or C₁₋₆alkyl.

Alkyl groups, whether alone or as part of another group, may be straight chain or branched. The term “C₁₋₆alkyl” refers to an alkyl group having from one to six carbon atoms, in any isomeric form, such as methyl, ethyl, propyl, isopropyl, butyl, isobutyl, sec-butyl, tert-butyl, pentyl, neopentyl, sec-pentyl, n-pentyl, isopentyl, tert-pentyl and hexyl.

The term “halogen” is used herein to describe, unless otherwise stated, fluorine, chlorine, bromine or iodine.

Where used herein the term “naphthyl” is intended, unless otherwise stated, to denote both naphth-1-yl and naphth-2-yl groups.

The term “heteroaryl” is intended to describe an aromatic or a benzofused aromatic ring containing 1 to 3 heteroatoms selected from oxygen, nitrogen and sulphur. Suitable examples of such aromatic rings include thienyl, furyl, pyrrolyl, triazolyl, imidazolyl, oxazolyl, thiazolyl, oxadiazolyl, isothiazolyl, isoxazolyl, thiadiazolyl, pyrazolyl, pyrimidyl, pyridazinyl, pyrazinyl and pyridyl. Suitable examples of such benzofused aromatic rings Include quinolinyl, isoquinolinyl, indolyl, benzofuryl, benzothienyl, benzimidazolyl, benzoxazolyl and the like.

The term “C₃₋₆cycloalkyl” refers to a cycloalkyl group consisting of from 3 to 6 carbon atoms, such as cyclopropane, cyclobutane, cyclopentane or cyclohexane.

The term “C₁₋₆alkoxy” refers to a straight chain or branched chain alkoxy (or “alkyloxy”.) group consisting of from one to six carbon atoms, such as methoxy, ethoxy, propoxy, isopropoxy, butoxy, isobutoxy, sec-butoxy, tert-butoxy, pentoxy, neopentoxy, sec-pentoxy, n-pentoxy, isopentoxy, tert-pentoxy and hexoxy.

The term “haloC₁₋₆alkyl” refers to a C₁₋₆alkyl group which is substituted by one or more halogens. Examples include CF₃.

The term “5-7 membered heterocyclic ring” is used herein to mean a non aromatic ring containing 1 to 3 heteroatoms selected from oxygen, nitrogen and sulphur. Suitable examples of such heterocyclic rings include piperidinyl, piperazinyl, pyrrolidinyl, imidazolidinyl, pyrazolidinyl, oxazolinyl, isothiazolidinyl, thiazolidinyl, dioxolanyl, thiazinanyl, dioxanyl and morpholinyl.

The heteroaryl and 5-7 membered heterocyclic rings, as described above, may be linked to the remainder of the molecule via a carbon atom or, when present, a suitable nitrogen atom.

Within the Definition of R^{a} Formula (i):

When P¹ is heteroaryl a preferred example is pyridyl. Preferably P¹ is phenyl.

When a is other than 0, preferred R¹ groups include halogen (particularly fluoro or chloro), C₁₋₆alkyl group (particularly methyl), CF₃ and cyano. When a is 2 or 3 the groups R¹ can be the same or different.

Preferably a is 1 or 2, most preferably 2.

Within the Definition of R^{a} Formula (ii):

When P³ is heteroaryl preferred examples include pyridyl and pyrazolyl. P³ is preferably phenyl.

P² is preferably phenyl, a heteroaryl group such as pyridyl, pyrazinyl, oxadiazolyl or oxazolyl or P² is a 5-7 membered heterocycle such as piperidinyl or piperazinyl.

When b is other than 0, preferred R² groups include halogen (particularly chloro), C₁₋₆alkyl group (particularly methyl), heteroaryl (particularly oxadiazolyl optionally substituted by C₁₋₆alkyl) or a 5-7 membered heterocyclic ring (particularly 2-oxo pyrrolidinyl). When b is 2 or 3 the groups R² may be the same or different. Preferably b is 0, 1 or 2.

When c is other than 0, preferred R³ groups are halogen (particularly chloro) and C₁₋₆alkyl group (particularly methyl). When c is 2 or 3 the groups R³ may be the same or different. Preferably c is 0 or 1.

A preferred group of formula (ii) is that in which P² is phenyl or pyridyl and P³ is phenyl or pyridyl. Such groups may be optionally substituted by the preferred R² and R³ groups as described above.

R^{b} is preferably hydrogen or halogen (particularly fluoro or chloro).

R^{c} is preferably hydrogen or methyl.

Preferred compounds of this invention are examples E1-E24 (as described below) or a pharmaceutically acceptable salt thereof.

The compounds of formula (I) can form acid addition salts thereof. It will be appreciated that for use in medicine the salts of the compounds of formula (I) should be pharmaceutically acceptable. Suitable pharmaceutically acceptable salts will be apparent to those skilled in the art and include those described in J. Pharm. Sci., 1977, 66, 1-19, such as acid addition salts formed with inorganic acids e.g. hydrochloric, hydrobromic, sulfuric, nitric or phosphoric acid; and organic acids e.g. succinic, maleic, acetic, fumaric, citric, tartaric, benzoic, p-toluenesulfonic, methanesulfonic or naphthalenesulfonic acid. Certain of the compounds of formula (I) may form acid addition salts with one or more equivalents of the acid. The present invention includes within its scope all possible stoichiometric and non-stoichiometric forms.

The compounds of formula (I) may be prepared in crystalline or non-crystalline form, and, if crystalline, may optionally be hydrated or solvated. This invention includes within its scope stoichiometric hydrates as well as compounds containing variable amounts of water and/or solvent.

Certain compounds of formula (I) are capable of existing in stereoisomeric forms (e.g. geometric (or “cis-trans”) isomers, diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The invention also extends to any tautomeric forms and mixtures thereof.

Compounds of the invention can be prepared using procedures known in the art. In a further aspect the present invention also provides a process for the preparation of a compound of formula (I) or a pharmaceutically acceptable salt thereof which comprises either

(a) when Y is NH, coupling a compound of formula (II):

R^{a}—NH₂ (II)

in which R^{a} is as defined in formula (I) with a compound of formula (III):

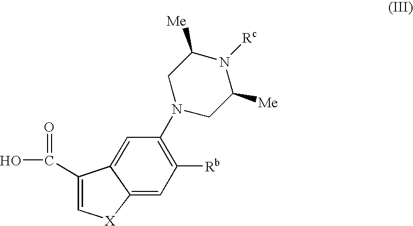

in which X, R^{b} and R^{c} are as defined in formula (I); or

(b) when Y is NH, coupling a compound of formula (II):

R^{a}—NH₂ (II)

in which R^{a} is as defined in formula (I) with a compound of formula (IV):

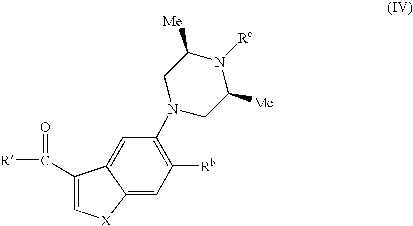

in which X, R^{b} and R^{c} are as defined in formula (I) and R′ is a C₁₋₆alkoxy group; or

(c) when Y=a single bond or CH₂, reacting a compound of formula (V);

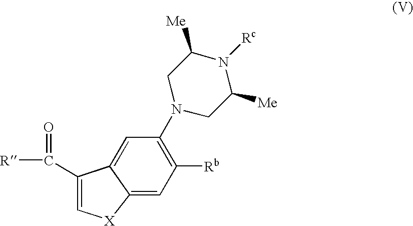

in which X, R^{b} and R^{c} are as defined in formula (I) and R″ is hydrogen, chloro, di-C₁₋₆alkylamino or C₁₋₆alkylC₁₋₆alkoxyamino with a compound of formula (VI)

R^{a}—(CH₂)_{q}-M (VI)

in which R^{a} is as defined for formula (I), q is 0 or 1 and M is Mg, Zn, Cd or Li; or

(d) reacting a compound of formula (II)

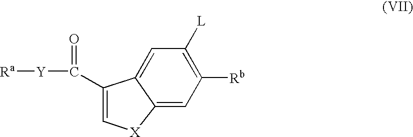

in which R^{a}, X, Y and R^{b} are as defined in formula (I) and L is a bromine or iodine with a compound of formula (VIII)

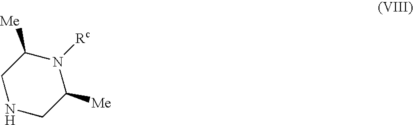

in which R^{c} is as defined in formula (I); or

(e) when X=NH and Y=a single bond or CH₂, reacting a compound of formula (IX):

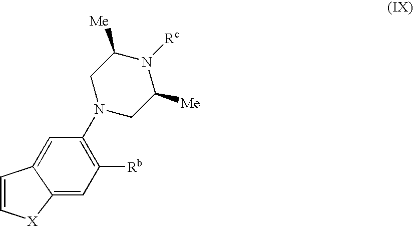

in which R^{b} and R^{c} are as defined in formula (I), with a compound of formula (X)

R^{a}—(CH₂)_{q}—COCl (X)

in which R^{a} is as defined for formula (I) and q is 0 or 1;

and optionally thereafter for either process (a), (b), (c), (d) or (e):

removing any protecting groups, and/or

converting a compound of formula (I) into another compound of formula (I), and/or

forming a pharmaceutically acceptable salt.

The reaction in process (a) is typically carried out in a solvent such as dichloromethane in the presence of 1-(3-dimethylaminopropyl)-3-ethylcarbodiimide and 1-hydroxybenzotriazole.

The reaction in process (b) is typically carried out in a solvent such as toluene at elevated temperature in the presence of trimethylaluminium.

The reaction in process (c) is typically carried out in a solvent such as diethyl ether or tetrahydrofuran. Additionally when R is H, the reaction is followed by oxidation with a reagent such as manganese dioxide.

The reaction in process (d) may be carried out in a solvent such as toluene or 1,4-dioxane in the presence of a base such as sodium tert-butoxide or cesium carbonate together with a palladium catalyst and a phosphine ligand. These type of reactions are described by S L Buchwald in Acc. Chem. Res. 1998, 31,805.

The reaction in process (e) may be carried out in a solvent such as diethyl ether, tetrahydrofuran or dichloromethane in the presence of a Grignard reagent such as ethylmagnesium bromide.

Compounds of formula (I) can be converted into further compounds of formula (I) using standard techniques. The following examples are given by way of illustration of this point rather than limitation. For compounds of formula (I) wherein R^{c} is hydrogen, it is possible to introduce a C₁₋₆alkyl group by conventional alkylation using 1 molar equivalent of a C₁₋₆alkyl halide and 1 molar equivalent of a suitable base in an Inert solvent.

Intermediate compounds of formula (II), (III), (IV), (V), (VI), (VII), (VIII), (IX) and (X) are either commercially available or can be prepared using methods described herein, by methods known to those skilled in the art or by analogous methods thereto.

It will be appreciated to those skilled in the art that It may be necessary to protect certain reactive substituents during some of the above procedures, such as in the preparation of indole examples when X is NH. Standard protection and deprotection techniques, such as those described in Greene T. W. Protective groups in organic synthesis, New York, Wiley (1981), can be used. For example, primary amines can be protected as phthalimide, benzyl, benzyloxycarbonyl or trityl derivatives. Carboxylic acid groups can be protected as esters. Aldehyde or ketone groups can be protected as acetals, ketals, thioacetals or thioketals. The indole NH group can be protected as a silyl derivative. Deprotection of such groups is achieved using conventional procedures well known in the art. For example, protecting groups such as t-butyloxycarbonyl may be removed using an acid such as hydrochloric or trifluroroacetic acid in a suitable solvent such as dichloromethane, diethylether, isopropanol or mixtures thereof.

Pharmaceutically acceptable salts may be prepared conventionally by reaction with the appropriate acid or acid derivative.

The involvement of serotonin (5-hydroxytryptamine; 5-HT) receptors in a number of pharmacological effects has been reviewed by R. A. Glennon in “Serotonin Receptors: Clinical Implications”, Neuroscience and Behavioural Reviews, 1990, 14, 35 and by L. O. Wilkinson and C. T. Dourish in “Serotonin Receptor Subtypes: Basic and Clinical Aspects” S. Peroutka Ed., John Wiley and Sons, New York, 1991 p.147.

Serotonin receptors have been implicated in pharmacological effects such as mood disorders including depression (both bipolar and unipolar), single or recurrent major depressive episodes with or without psychotic features, catatonic features, melancholic features, a typical features or postpartum onset, depression resulting from a general medical condition including, but not limited to, myocardial infarction, diabetes, miscarriage or abortion, etc., seasonal affective disorder and dysthymia, anxiety disorders, including generalised anxiety and social anxiety disorder, panic disorder, agoraphobia, social phobia, obsessive compulsive disorder and post-traumatic stress disorder; memory disorders, including dementia, amnesic disorders and age-associated memory impairment; disorders of eating behaviours, including anorexia nervosa and bulimia nervosa, sleep disorders (including narcolepsy, dyssomnia, insomnia, sleep apnea and disturbances of circadian rhythm), motor disorders such as Parkinson's disease, dementia in Parkinson's disease, neuroleptic-induced Parkinsonism and tardive dyskinesias, pain disorders (particularly neuropathic pain), as well as other psychiatric disorders such as schizophrenia and psychosis. Serotonin receptor ligands have been shown to be of use in the treatment of emesis and nausea and may also be of use in endocrine disorders such as hyperlactinaemia, vasospasm (particularly in the cerebral vasculature), cerebellar ataxia and hypertension, as well as disorders of the gastrointestinal tract where changes in motility and secretion are involved such as irritable bowel syndrome, and in treatment of withdrawal symptoms from abuse of drugs such as of cocaine, ethanol, nicotine, benzodiazepines, alcohol, caffeine, phencyclidine (phencyclidine-like compounds), opiates (e.g. cannabis, heroin, morphine), sedative ipnotic, amphetamine or amphetamine-related drugs (e.g. dextroamphetamine, methylamphetamine) or a combination thereof. They may also be of use in the treatment of pre-menstrual tension, sexual dysfunction and hypothermia.

Ligands with high affinity for the 5-HT₁ receptors are well recognised as having therapeutic utility for the treatment of the above conditions. It has been suggested that a selective 5-HT_{1B} receptor antagonist should act as a fast onset antidepressant (P. Blier Trends Pharmacol. Sci. 1994, 15, 220).

Accordingly, the present invention provides a compound of formula (I) or a pharmaceutically acceptable salt for use in therapy.

In particular, the present invention provides for a compound of formula (I) or a pharmaceutically acceptable salt for use in the treatment of depression (which includes bipolar depression, unipolar depression, single or recurrent major depressive episodes with or without psychotic features, catatonic features, melancholic features, a typical features or postpartum onset, depression resulting from a general medical condition including, but not limited to, myocardial infarction, diabetes, miscarriage or abortion, etc., seasonal affective disorder and dysthymia), anxiety disorders including generalised anxiety and social anxiety disorder, panic disorders, schizophrenia, psychosis, agoraphobia, social phobia, obsessive compulsive disorder and post-traumatic stress disorder; memory disorders, including dementia, amnesic disorders and age-associated memory impairment; disorders of eating behaviours, including anorexia nervosa and bulimia nervosa; sleep disorders (including narcolepsy, dyssomnia, insomnia, sleep apnea and disturbances of circadian rhythm); motor disorders such as Parkinson's disease, dementia in Parkinson's disease, neuroleptic-induced Parkinsonism and tardive dyskinesias; pain disorders (particularly neuropathic pain); emesis and nausea; endocrine disorders such as hyperlactinaemia; vasospasm (particularly in the cerebral vasculature); cerebellar ataxia; hypertension; gastrointestinal disorders where changes in motility and secretion are involved, such as irritable bowel syndrome; treatment of withdrawal symptoms from abuse of drugs such as of cocaine, ethanol, nicotine, benzodiazepines, alcohol, caffeine, phencyclidine (phencyclidine-like compounds), opiates (e.g. cannabis, heroin, morphine), sedative ipnotic, amphetamine or amphetamine-related drugs (e.g. dextroamphetamine, methylamphetamine) or a combination thereof; premenstrual tension; sexual dysfunction and hypothermia. In particular, the present Invention provides for a compound of formula (I) or a pharmaceutically acceptable salt for use in the treatment of depression.

It is to be understood that the term “treatment” as used herein includes prophylaxis as well as alleviation of established symptoms.

In a further aspect the invention provides a method of treating a disorder where an antagonist of the 5-HT_{1B} receptor is beneficial, particularly the aforementioned disorders, particularly depression, which comprises administering a safe and therapeutically effective amount of compound of formula (I) or a pharmaceutically acceptable salt to a patient in need thereof.

In another aspect, the invention provides the use of a compound of formula (I) or a pharmaceutically acceptable salt thereof in the manufacture of a medicament for the treatment a disorder in which an antagonist of the 5-HT_{1B} receptor is beneficial, particularly the aforementioned disorders, more particularly depression.

The affinities of the compounds of this invention for the 5-HT_{1B} receptor can be determined by the following radioligand binding assay. CHO cells expressing 5-HT_{1B} receptors (4×10⁷ cells/ml) are homogenised in Tris buffer Mg²⁺ and stored in 1.0 ml aliquots. 0.4 ml of a cell suspension is incubated with [³H]-5-HT (4 nM) in Tris Mg HCl buffer (pH 7.7) and test drug, at 37° C. for 45 minutes. Each test drug is tested at 10 concentrations (0.01 mM to 0.3 nM final concentration), with non-specific binding defined using 0.01 mM 5-HT. The total assay volume is 0.5 ml. Incubation is stopped by rapid filtration using a Tomtec Harvester (filters prewashed in 0.3% polyethylenimine) and radioactivity measured by Topcount scintillation counting. pKi values are calculated from the IC₅₀ generated by an iterative least squares curve fitting programme.

All examples tested in accordance with this radioligand binding assay were found to have a pKi>7.0 at 5-HT_{1B} receptors with some having a pKi>8.0.

The selectivity of the compounds of this invention for 5-HT_{1B} receptors can be determined using binding assay methods which are well known to those skilled in the art. The majority of examples tested were found to have a greater than a 10-fold selectivity over 5-HT_{1D} receptors and over other binding sites within the CNS, in particular, other 5-HT receptor sub-types and dopaminergic receptors.

The intrinsic activity of the compounds of this invention can be determined according to the following procedure. CHO cell membranes stably expressing human 5-HT_{1B} receptors are homogenised in HEPES/EDTA buffer and stored in 1 ml aliquots, and [³⁵S]GTPγS binding studies are carried out essentially as described by Lazareno et al., (Life Sci., 1993, 52, 449) with some minor modifications. Membranes from 10⁶ cells are pre-incubated at 30° C. for 30 minutes in 20 mM HEPES buffer (pH 7.4) in the presence of MgCl₂ (3 mM), NaCl (100 mM), GDP (10 μm) and ascorbate (0.2 mM), with or without compounds. The reaction is started by the addition of 50 μl of [³⁵S]GTPγS (100 μm, assay concentration) followed by a further 30 minutes incubation at 30° C. Non-specific binding was determined using non-radiolabelled GTPYS (20 μM) added prior to the membranes. The reaction is terminated by rapid filtration through Whatman GF/B grade filters followed by 5×1 ml washes with ice cold HEPES (20 mM)/MgCl₂ (3 mM) buffer. Radioactivity is measured using liquid scintillation spectrometry. This procedure is hereafter referred to as the [³⁵S]GTPγS functional assay.

It has been found, using the [³⁵S]GTPγS functional assay, that certain compounds of formula (I) show varying levels of intrinsic efficacy, which is defined by a scale in which the value 1.0 defines the maximum response elicited by the agonist 5-HT, 0.0 defines antagonism and a negative value indicates inverse agonism. The difficulties in describing intrinsic activity of drugs acting at G protein coupled receptors is recognised in the art (Hoyer and Boddeke, Trends in Pharmacological Sciences, July 1993, [Vol. 14], page 270-275). We believe that however these ligands are classified according to this functional assay, the compounds of this invention will be useful antidepressants in vivo. It is believed that the preferred compounds of this invention will display 5-HT_{1B} antagonist activity in vivo and that such compounds will have a rapid onset of action. A rapid onset of action is particularly advantageous for antidepressant compounds: by ‘rapid onset of action’ we mean that a therapeutic response is seen within 7 days from first administration of the compound, as opposed to a period of about 21 days or more which is typical of SSRI's, tricyclic antidepressants and buspirone.

Compounds of formula (I) which have an intrinsic activity of 0.5 or less in the in vitro [³⁵S]GTPγS functional assay are preferred, as these compounds are more likely to be full antagonists in vivo. Particularly preferred compounds of this invention have an intrinsic activity in the range 0.0-0.3 or are inverse agonists in this functional assay.

It has been found that the compounds of this invention have a particularly advantageous profile in that they demonstrate high affinity and selectivity for the 5-HT_{1B} receptor together with low intrinsic activity in the [³⁵S]GTPγS functional assay.

It will be appreciated by those skilled in the art that the compounds according to the invention may advantageously be used in conjunction with one or more other therapeutic agents, for instance, different antidepressant agents such as 5HT3 antagonists, serotonin agonists, NK-1 antagonists, selective serotonin reuptake inhibitors (SSRI), noradrenaline re-uptake inhibitors (SNRI), tricyclic antidepressants and/or dopaminergic antidepressants.

Suitable 5HT3 antagonists which may be used in combination of the compounds of the inventions include for example ondansetron, granisetron, metoclopramide.

Suitable serotonin agonists which may be used in combination with the compounds of the invention include sumatriptan, rauwolscine, yohimbine, metoclopramide.

Suitable SSRIs which may be used in combination with the compounds of the invention include fluoxetine, citalopram, femoxetine, fluvoxamine, paroxetine, indalpine, sertraline, zimeldine.

Suitable SNRIs which may be used in combination with the compounds of the invention include venlafaxine and reboxetine.

Suitable tricyclic antidepressants which may be used in combination with a compound of the invention include imipramine, amitriptiline, chlomipramine and nortriptiline.

Suitable dopaminergic antidepressants which may be used in combination with a compound of the invention include bupropion and amineptine.

It will be appreciated that the compounds of the combination or composition may be administered simultaneously (either in the same or different pharmaceutical formulations), separately or sequentially.

In order to use the compounds of formula (I) in therapy, they will normally be formulated into a pharmaceutical composition in accordance with standard pharmaceutical practice. The present invention also provides a pharmaceutical composition, which comprises a compound of formula (I) or a pharmaceutically acceptable salt thereof, and a pharmaceutically acceptable carrier or excipient.

In a further aspect, the present invention provides a process for preparing a pharmaceutical composition, the process comprising mixing a compound of formula (I) or a pharmaceutically acceptable salt thereof and a pharmaceutically acceptable carrier or excipient.

A pharmaceutical composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents (e.g. pregelatinised maize starch, polyvinylpyrrolidone or hydroxypropyl methylcellulose), fillers (e.g. lactose, microcrystalline cellulose or calcium hydrogen phosphate), tabletting lubricants (e.g. magnesium stearate, talc or silica), disintegrants (e.g. potato starch or sodium starch glycollate) and acceptable wetting agents (e.g. sodium lauryl sulphate). The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents (e.g. sorbitol syrup, cellulose derivatives or hydrogenated edible fats), emulsifying agents (e.g. lecithin or acacia), non-aqueous vehicles (which may include edible oils eg. almond oil, oily esters, ethyl alcohol or fractionated vegetable oils), preservatives (e.g. methyl or propyl-p-hydroxybenzoates or sorbic acid), and, if desired, conventional flavourings or colorants, buffer salts and sweetening agents as appropriate. Preparations for oral administration may be suitably formulated to give controlled release of the active compound.

For parenteral administration, fluid unit dosage forms are prepared utilising a compound of the invention or pharmaceutically acceptable salt thereof and a sterile vehicle. Formulations for injection may be presented in unit dosage form e.g. in ampoules or in multi-dose, utilising a compound of the invention or pharmaceutically acceptable salt thereof and a sterile vehicle, optionally with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. Advantageously, a surfactant or wetting agent is included in the composition to facilitate uniform distribution of the compound.

Lotions may be formulated with an aqueous or oily base and will in general also contain one or more emulsifying agents, stabilising agents, dispersing agents, suspending agents, thickening agents, or colouring agents. Drops may be formulated with an aqueous or non-aqueous base also comprising one or more dispersing agents, stabilising agents, solubilising agents or suspending agents. They may also contain a preservative.

The compounds of the invention may also be formulated in rectal compositions such as suppositories or retention enemas, e.g. containing conventional suppository bases such as cocoa butter or other glycerides.

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of the invention may be formulated as solutions for administration via a suitable metered or unitary dose device or alternatively as a powder mix with a suitable carrier for administration using a suitable delivery device. Thus compounds of formula (I) may be formulated for oral, buccal, parenteral, topical (including ophthalmic and nasal), depot or rectal administration or in a form suitable for administration by inhalation or insufflation (either through the mouth or nose).

The compounds of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

The composition may contain from 0.1% to 99% by weight, preferably from 10 to 60% by weight, of the active material, depending on the method of administration.

The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 200 mg, and such unit doses may be administered more than once a day, for example two or three times a day. Such therapy may extend for a number of weeks or months.

All publications, including but not limited to patents and patent applications, cited in this specification are herein incorporated by reference as if each individual publication were specifically and individually indicated to be incorporated by reference herein as though fully set forth.

The following descriptions and Examples illustrate the preparation of compounds of the invention.

Description 1

Diethyl 3-(4-bromophenoxy)-2-oxosuccinate (D1)

A stirred suspension of NaOEt (5.4 g, 80 mmole) in dry Et₂O (150 ml) at room temp. under argon was treated dropwise over 5 min with a solution of diethyl oxalate (10.5 ml, 78 mmole) in Et₂O (15 ml) and maintained for 20 min, then ethyl (4-bromophenoxy)acetate (19.4 g, 75 mmole) was added portionwise over 15 min and the mixture stirred at room temp. for 2 h. It was added to water (150 ml) with stirring and the aqueous layer separated. The Et₂O layer was extracted with 1 M NaOH solution (2×100 ml) and the combined aqueous solutions acidified with conc. HCl acid and extracted with EtOAc. The extract was dried (Na₂SO₄) and concentrated under vacuum to afford the title compound as a yellow oil (23.5 g, 87%).

¹H NMR highly complex due to probable mixture of keto and enol forms

Description 2

Diethyl 5-bromobenzofuran-2,3-dicarboxylate (D2)

Stirred conc H₂SO₄ acid (200 ml) at room temp. under argon was treated over 15 min with D1 (23.5 g, 65 mmole) and maintained for 6 h. The mixture was poured cautiously into well stirred ice/water (1000 ml) and extracted with EtOAc. The extract was washed with 1M NaOH solution, dried (Na₂SO₄) and concentrated under vacuum to afford the title compound as a yellow oil (17.6 g, 79%).

¹H NMR (250 MHz, CDCl₃) δ 8.08 (d, 1H), 7.58 (dd, 1H), 7.47 (d, 1H), 4.50 (q, 2H), 4.48 (q, 2H), 1.45 (t, 3H), 1.44 (t, 3H).

Description 3

5-Bromobenzofuran-2,3-dicarboxylic Acid (D3)

A stirred solution of D2 (17.6 g, 52 mmole) in EtOH (100 ml) was treated with 2M NaOH solution (200 ml) and heated at reflux under argon for 2 h, then allowed to cool and concentrated under vacuum to approx. 200 ml. The aqueous mixture was washed with Et₂O, then added to excess conc. HCl acid and the resultant precipitate filtered off, washed with water and dried to afford the title compound as a white solid (12.2 g, 82%).

¹H NMR (250 MHz, d⁶DMSO) δ 13.4 (br s, 2H), 8.25 (d, 1H), 7.78 (d, 1H), 7.68 (dd, 1H).

Description 4

5-Bromobenzofuran-3-carboxylic Acid (D4)

Finely powdered D3 (9.7 g, 34 mmole) under argon was heated with gentle stirring up to its melting point (>250° C.) at which point the dark brown oil was observed to undergo decarboxylation. Heating was maintained until gas evolution ceased (0.25 h), then the mixture was allowed to cool. The residue was treated with 1M NaOH solution (120 ml) and Et₂O (100 ml) and stirred well for 0.5 h. The aqueous layer was isolated, filtered, then acidified with conc. HCl acid and extracted with EtOAc. The extract was dried (Na₂SO₄) and concentrated under vacuum to afford the title compound as a pale yellow solid (5.9 g, 72%).

¹H NMR (250 MHz, d⁶DMSO) δ 13.23 (br s, 1H), 8.75 (s, 1H), 8.09 (d, 1H), 7.72 (d, 1H), 7.58 (dd, 1H).

Description 5

N,N-Dimethyl-5-bromobenzofuran-3-carboxamide (D5)

A stirred suspension of D4 (3.5 g, 14 mmole) in DCM (100 ml) was treated with oxalyl chloride (1.9 ml, 22 mmole) and DMF (3 drops) and maintained at room temp. for 3 h, then concentrated under vacuum to leave the acid chloride as a yellow solid. This was redissolved in DCM (100 ml) and added dropwise over 10 min to a stirred solution of dimethylamine (11 ml of 2M solution in THF, 22 mmole) and pyridine (1.8 ml, 22 mmole) in DCM (60 ml) at 0° C. under argon. The mixture was allowed to warm to room temp. over 1.5 h, then washed with 1M HCl acid (100 ml) and brine, dried (Na₂SO₄) and concentrated under vacuum. The residue was chromatographed on silica gel eluting with 1:1′ EtOAc:60-80 petrol to afford the title compound as a pale yellow solid (3.25 g, 81%). MH⁺ 268/270.

¹H NMR (250 MHz, CDCl₃) δ 7.93 (d, 1H), 7.84 (s, 1H), 7.46 (dd, 1H), 7.40 (d, 1H), 3.16 (s, 6H).

Description 6

cis-N,N-Dimethyl-5-(4-benzyl-3,5-dimethylpiperazin-1-yl)benzofuran-3-carboxamide (D6)

A mixture of tris(dibenzylideneacetone)dipalladium(0) (110 mg, 0.12 mmole) and BINAP (225 mg, 0.36 mmole) in dry toluene (50 ml) under argon was stirred at room temp. for 0.5 h, then treated succesively with a solution of D5 (3.2 g, 12 mmole) in dry toluene (50 ml), cis-1-benzyl-2,6-dimethylpiperazine (Org. Prep. Proc. 1976, 8, 19) (3.7 g, 18 mmole) and sodium tert-butoxide (1.9 g, 20 mmole). The mixture was heated at 100° C. for 24 h, then allowed to cool and concentrated under vacuum. The residue was shaken well with EtOAc and 1 M HCl acid. The acid layer was isolated, basified by addition of K₂CO₃ and extracted with EtOAc. The extract was dried (Na₂SO₄), concentrated under vacuum and the residue chromatographed on silica gel eluting with 0-50% EtOAc/Et₂O to afford the title compound as a pale yellow oil (2.7 g, 58%). MH⁺ 392.

¹H NMR (250 MHz, CDCl₃) δ 7.77 (s, 1H), 7.45-7.20 (m, 7H), 7.04 (dd, 1H), 4.13 (s, 2H), 3.44 (brd, 2H), 3.17 (s, 6H), 2.93-2.80 (m, 2H), 2.60 (t, 2H), 1.13 (d, 6H).

Description 7

cis-N,N-Dimethyl-5-(3,5-dimethylpiperazin-1-yl)benzofuran-3-carboxamide (D7)

A stirred solution of D6 (0.45 g, 1.1 mmole) in EtOH (50 ml) was treated with 10% Pd—C (100 mg of dry powder) and cyclohexene (5 ml) and heated under argon for 3 h. The mixture was allowed to cool, filtered through Kieselguhr and the filtrate concentrated under vacuum to afford the title compound as/an orange gum (0.33 g, 95%).

¹H NMR (250 MHz, CDCl₃) δ 7.78 (s, 1H), 7.40 (d, 1H), 7.29 (d, 1H), 7.05 (dd, 1H), 4.60 (br s, 1H), 3.51 (br d, 2H), 3.33-3.18 (m, 2H), 3.17 (s, 6H), 2.65 (t, 2H), 1.35 (d, 6H).

Description 8

cis-N,N-Dimethyl-5-(3,4,5-trimethylpiperazin-1-yl)benzofuran-3-carboxamide (D8)

A stirred solution of D7 (0.33 g, 1.1 mmole) in MeOH (10 ml) was treated with formaldehyde (0.55 ml of 37% w/v aqueous solution, 4.0 mmole) followed by portionwise addition over 5 min of NaCNBH₃ (0.13 g, 2.2 mmole) and the mixture maintained at room temp. for 3 h, then concentrated under vacuum. The residue was treated with 10% Na₂CO₃ solution and extracted with EtOAc. The extract was dried (Na₂SO₄), concentrated under vacuum and the residue chromatographed on neutral alumina eluting with EtOAc to afford the title compound as a pale yellow oil (0.22 g, 61%). MH⁺ 316.

¹H NMR (250 MHz, CDCl₃) δ 7.78 (s, 1H), 7.41 (d, 1H), 7.23 (d, 1H), 7.04 (dd, 1H), 3.46 (br d, 2H), 3.18 (s, 6H), 2.62 (t, 2H), 2.54-2.40 (m, 2H), 2.35 (s, 3H), 1.20 (d, 6H).

Description 9

cis-Methyl 5-(3,4,5-trimethylpiperazin-1-yl)benzofuran-3-carboxylate (D9)

A solution of D8 (0.19 g, 0.63 mmole) in EtOH (7 ml) was treated with 1M NaOH solution (7 ml) and heated at reflux under argon for 8 h, then concentrated under vacuum to leave an orange semi-solid. This was dissolved in MeOH (25 ml), treated with conc. H₂SO₄ acid (2 ml) and heated at reflux under argon for 8 h. The mixture was concentrated under vacuum, the residue treated with excess 10% Na₂CO₃ solution and extracted with EtOAc. The extract was dried (Na₂SO₄) and concentrated under vacuum to afford the title compound as an orange oil (0.16 g, 84%).

¹H NMR (250 MHz, CDCl₃) δ 8.18 (s, 1H), 7.54 (d, 1H), 7.40 (d, 1H), 7.04 (dd, 1H), 3.93 (s, 3H), 3.47 (dd, 2H), 2.62 (t, 2H), 2.50-2.38 (m, 2H), 2.34 (s, 3H), 1.19 (d, 6H).

Description 10

cis-Methyl 5-(3,5-dimethylpiperazin-1-yl)benzofuran-3-carboxylate (D10)

The title compound was prepared from D6, following a similar procedure to Description 9 followed by Description 7, as a yellow oil.

¹H NMR (250 MHz, CDCl₃) δ 8.19 (s, 1H), 7.56 (d, 1H), 7.41 (d, 1H), 7.05 (dd, 1H), 3.93 (s, 3H), 3.52 (br d, 2H), 3.20-3.05 (m, 2H), 2.34 (t, 2H), 1.16 (d, 6H). NH not discernible.

Description 11

N-[tert-Butoxycarbonyl]-2′-methyl-4′-(5-methyl-1,2,4-oxadiazol-3-yl)-4-biphenylamine (D11)

A stirred suspension of 2′-methyl-4′-(5-methyl-1,2,4-oxadiazol-3-yl)biphenyl-4 carboxylic acid (EP0533268A1) (3.0 g, 10 mmole) in tert-butanol (60 ml) under argon was treated with diphenylphosphoryl azide (3.3 g, 12 mmole) and heated under reflux for 6 h, then concentrated under vacuum and the residue treated with 10% Na₂CO₃ solution and extracted with EtOAc. The extract was dried (Na₂SO₄), concentrated under vacuum and the residue chromatographed on silica gel eluting with 30% EtOAc/60-80 petrol to afford the title compound as a white solid (1.70 g, 46%). ¹H NMR (250 MHz, CDCl₃) δ 7.96 (s, 1H), 7.91 (d, 1H), 7.44 (d, 2H), 7.35-7.23 (m, 3H), 6.61 (s, 1H), 2.67 (s, 3H), 2.34 (s, 3H), 1.54 (s, 9H).

Description 12

2′-Methyl-4(5-methyl-11,2,4-oxadiazol-3-yl)₄-biphenylamine (D12)

A solution of D11 (1.70 g, 4.6 mmole) In THF (50 ml) was treated with 2M HCl acid (20 ml) and heated at reflux for 8 h, then concentrated under vacuum to approx. 20 ml volume. The aqueous residue was basified with K₂CO₃ and extracted with EtOAc. The extract was dried (Na₂SO₄), concentrated under vacuum and the residue chromatographed on silica gel eluting with 40% EtOAc/60-80 petrol to afford the title compound as a beige solid (0.98 g, 80%).

¹H NMR (250 MHz, CDCl₃) δ 7.96 (d, 1H), 7.90 (dd, 1H), 7.32 (d, 1H), 7.17 (d, 2H), 6.75 (d, 2H), 3.75 (br s, 2H), 2.67 (s, 3H), 2.37 (s, 3H).

Description 13

Ethyl (4-bromo-3-fluorophenoxy)acetate (D13)

A stirred solution of 4-bromo-3-fluorophenol (preparation 3.1 in WO 99/43763) (14.7 g, 77 mmole) in acetone (200 ml) was treated with K₂CO₃ (15.2 g, 110 mmole) and ethyl bromoacetate (13.1 g, 78 mmole) and heated at reflux for 6 h. The mixture was concentrated under vacuum and the residue treated with water (200 ml) and extracted with EtOAc. The extract was dried (Na₂SO₄) and concentrated under vacuum to afford the title compound as a white solid (21.7 g, 100%).

¹H NMR (250 MHz, CDCl₃) δ 7.42 (dd, 1H), 6.71 (dd, 1H), 6.62 (ddd, 1H), 4.59 (s, 2H), 4.27 (t, 2H), 1.30 (t, 3H).

Description 14

cis-N,N-Dimethyl-5-(4-benzyl-3,5-dimethylpiperazin-1-yl)-6-fluorobenzofuran-3-carboxamide (D14)

The title compound was prepared as a pale yellow oil from ethyl (4-bromo-3-fluorophenoxy)acetate (D13) following similar procedures to Descriptions 1-6. MH⁺ 410.

¹H NMR (250 MHz, CDCl₃) δ 7.77 (s, 1H), 7.45-7.18 (m, 7H), 3.90 (s, 2H), 3.27 (br, d, 2H), 3.19 (s, 6H), 3.00-2.85 (m, 2H), 2.59 (t, 2H), 1.11 (d, 6H).

Description 15

cis-Methyl 6-fluoro-5-(3,4,5-trimethylpiperazin-1-yl)benzofuran-3-carboxylate (D15)

This was prepared as a yellow oil from cis-N,N-dimethyl-5-(4-benzyl-3,5-dimethylpiperazin-1-yl)-6-fluorobenzofuran-3-carboxamide (D14) following similar procedures to Descriptions 7-9.

¹H NMR (250 MHz, CDCl₃) δ 8.18 (s, 1H), 7.59 (d, 1H), 7.24 (d, 1H), 3.93 (s, 3H), 3.30 (d, 2H), 2.64 (t, 2H), 2.59-2.45 (m, 2H), 2.36 (s, 3H), 1.18 (d, 6H).

Description 16

cis-Methyl 5-(3,5-dimethylpiperazin-1-yl)-6-fluorobenzofuran-3-carboxylate (D16)

This was prepared as a beige solid from cis-N,N-dimethyl-5-(4-benzyl-3,5-dimethylpiperazin-1-yl)-6-fluorobenzofuran-3-carboxamide (D14) following similar procedures to Description 9 followed by Description 7.

¹H NMR (250 MHz, CDCl₃) δ 8.18 (s, 1H), 7.61 (d, 1H), 7.25 (d, 1H), 3.94 (s, 3H), 3.33 (d, 2H), 3.25-3.09 (m, 2H), 2.35 (t, 2H), 1.14 (d, 6H). NH not discernible.

Description 17

N,N-Dimethyl-5-bromobenzothiophene-3-carboxamide (D17)

The title compound was prepared from 5-bromobenzothiophene-3-carboxylic acid (J. Chem. Soc. 1967, 20, 2084) using a similar procedure to Description 5 as a colourless oil (97%).

¹H NMR (250 MHz, CDCl₃) δ 8.01 (d, 1H), 7.72 (d, 1H), 7.58 (s, 1H), 7.50 (dd, 1H), 2.97 (br s, 6H).

Description 18

cis-N,N-Dimethyl-5-(3,5-dimethylpiperazin-1-yl)benzothiophene-3-carboxamide (D18)

A mixture of palladium (II) acetate (0.70 g, 3.1 mmole), BINAP (0.36 g, 0.58 mmole) and cesium carbonate (1.87 g, 5.7 mmole) in dry 1,4-dioxane (20 ml) under argon was sonicated at 40° C. for 40 min., then treated succesively with D18 (1.0 g, 3.6 mmole) and cis-2,6-dimethylpiperaizine (0.64 g, 5.6 mmole). The mixture was heated at reflux for 24 h, then allowed to cool and concentrated under vacuum. The residue was treated with 10% K₂CO₃ solution and extracted with EtOAc. The extract was dried (Na₂SO₄), concentrated under vacuum and the residue chromatographed on silica gel eluting with MeOH/DCM to afford the title compound as a brown glass (0.72 g, 65%). MH⁺ 318.

¹H NMR (250 MHz, CDCl₃) δ 7.72 (d, 1H), 7.49 (s, 1H), 7.28 (t, 1H), 7.11 (dd, 1H), 3.55 (d, 2H), 3.10 (m, 8H), 2.38 (t, 2H), 1.18 (d, 6H). NH not discernible.

Description 19

cis-N,N-Dimethyl-5-(3,4,5-trimethylpiperazin-1-yl)benzothiophene-3-carboxamide (D19)

The title compound was prepared from D18 using a similar procedure to Description 8 as a brown glass (89%). MH⁺ 332.

¹H NMR (250 MHz, CDCl₃) δ 7.70 (d, 1H), 7.47 (s, 1H), 7.26 (m, 1H), 7.10 (dd, 1H), 4.24 (s, 1H), 3.46 (s, 2H), 3.14 (br s, 6H), 2.63 (t, 2H), 2.46 (m, 2H), 2.40 (s, 3H), 1.20 (d, 6H).

Description 20

cis-Methyl-5-(3,4,5-trimethylpiperazin-1-yl)benzothiophene-3-carboxylate (D20)

The title compound was prepared from D19 using a similar procedure to Description 9 as a white crystalline solid (82%).

¹H NMR (400 MHz, CDCl₃) δ 8.32 (s, 1H), 8.10 (d, 1H), 7.71 (d, 1H), 7.13 (dd, 1H), 3.93 (s, 3H), 3.58 (d, 2H), 2.66 (t, 2H), 2.43 (m, 2H), 2.33 (s, 3H), 1.21 (d, 6H).

Description 21

cis-Methyl-5-(3,5-dimethylpiperazin-1-yl)benzothiophene-3-carboxamide (D21)

The title compound was prepared from D18 using a similar procedure to Description 9 as brown glass (77%). MH⁺ 305.

¹H NMR (250 MHz, CDCl₃) δ 8.43 (s, 1H), 8.12 (d, 1H), 7.72 (d, 1H), 7.14 (dd, 1H), 3.94 (s, 3H), 3.60 (d, 2H), 3.13 (m, 2H), 2.34 (t, 2H), 1.18 (d, 6H).

Description 22

5-Amino-2-(4-cyanophenyl)-3-methylpyridine (D22)

A stirred solution of 5-amino-2-chloro-3-methylpyridine (220 mg, 1.5 mmole) and Na₂CO₃ (640 mg, 6.0 mmole) in 1,2-dimethoxyethane (20 ml) and water (15 ml) was degassed by bubbling argon through for 0.25 h, then 4-cyanophenylboronic acid (290 mg, 2.0 mmole) and tetrakis(triphenylphosphine)palladium (0) (75 mg, 0.067 mmole) were added and the mixture heated at 90° C. for 18 h. The solution was cooled, concentrated to 50% volume, then diluted with water (20 ml) and extracted with EtOAc (30 ml). The extract was shaken with 1 M HCl acid (20 ml), then the acid layer separated, basified by addition of solid K₂CO₃ and extracted with EtOAc. The extract was dried (Na₂SO₄), concetrated under vacuum and the residue chromatographed on silica gel eluting with 1:1 EtOAc/Et₂O to afford the title compound as a beige solid (55 mg, 17%). MH⁺ 210.

¹H NMR (250 MHz, CDCl₃) δ 8.03 (d, 1H), 7.71 and 7.62 (AB, 4H), 6.90 (d, 1H), 3.77 (br s, 2H), 2.98 (s, 3H).

Description 23

1-Acetyl-5-(3,5-dimethylpiperazin-1-yl)indoline (D23)

The title compound was prepared from 1-acetyl-5-bromoindoline using a similar procedure to D18 as a yellow solid (92%). MH⁺ 274.

¹H NMR (250 MHz, CDCl₃) δ 8.10 (d, 1H), 6.76-6.81 (m, 2H), 4.04 (t, 2H), 3.47 (d, 1H), 3.42 (s, 1H), 3.17 (t, 2H), 3.07 (m, 2H), 2.26 (m, 2H), 2.21 (s, 3H), 1.15 (d, 6H). NH not observed.

Description 24

1-Acetyl-5-(3,4,5-trimethylpiperazin-1-yl)indoline (D24)

The title compound was prepared from D23 following a similar procedure to Description 8, then recrystallised from EtOAc to afford a yellow solid (77%). MH⁺ 288.

¹H NMR (250 MHz, CDCl₃) δ 7.93 (d, 1H), 6.57-6.62 (m, 2H), 3.86 (t, 2H), 3.22 (br d, 2H), 3.00 (t, 2H), 2.38 (t, 2H), 2.25 (m, 2H), 2.16 (s, 3H), 2.04 (s, 3H), 1.00 (d, 6H).

Description 25

5-(3,4,5-Trimethylpiperazin-1-yl)indole (D25)

A solution of D24 (7.47 g, 0.026 mole) in 2M HCl (100 ml) was heated at reflux under argon for 2 h. On cooling, the mixture was basified (K₂CO₃), extracted (DCM×2), dried (Na₂SO₄) and evaporated in vacuo to a pale yellow oil, which was dissolved in toluene (100 ml), treated with 10% Pd—C (600 mg) and heated at reflux for 16 h. Further catalyst (600 mg) was added and heating continued for 16 h to completion. The mixture was filtered through kieselguhr, the residue chromatographed on silica gel eluting with 5% MeOH/DCM to afford a yellow solid (3.18 g, 50%). MH⁺ 244

¹H NMR (250 MHz, CDCl₃) δ 8.07 (br s, 1H), 7.25 (m, 1H), 7.14 (m, 2H), 6.95 (dd, 1H), 6.44 (m, 1H), 3.37 (dd, 2H), 2.59 (t, 2H), 2.40-2.51 (m, 2H), 2.33 (s, 3H), 1.15 (d, 6H).

Description 26

4-(Imidazol-1-yl)-3-methylaniline (D26)

A stirred mixture of 1-(2-methyl-4-nitrophenyl)-1H-imidazole (J. Med. Chem. 1994, 37(4), 467) (1.28 g, 6.3 mmole), cyclohexene (3 ml) and 10% palladium on carbon (50 mg) was heated to reflux for 18 h. The cooled mixture was filtered, and the filtate concentrated to dryness. The residue was triturated in Et₂O/hexane to afford the title compound as a buff powder (0.98 g, 90%). MH⁺ 174 1H NMR (250 MHz, CDCl₃) δ 7.51 (s, 1H), 7.16 (s, 1H), 6.98 (d, 2H), 6.52-6.60 (m, 2H), 3.76 (br s, 2H), 2.04 (s, 3H).

Description 27

1-(2-Methyl-4-nitrophenyl)piperazine (D27)

A stirred mixture of 2-fluoro-5-nitrotoluene (0.75 g, 4.8 mmole), piperazine (0.54 g, 6.2 mmole), potassium carbonate (0.99 g, 7.2 mmole) and tetrabutylammonium iodide (17 mg, 0.05 mmole) in DMSO (5 ml) was heated to 120° C. for 5 h. The cooled mixture was then poured into water (50 ml) and extracted (DCM×2). The extract was dried (Na₂SO₄), concentrated in vacuo and the residue chromatographed on silica gel eluting with DCM/MeOH/NH₄OH (100:10:1) to afford the title compound as yellow solid (0.77 g, 72%). MH⁺ 222.

¹H NMR (250 MHz, CDCl₃) δ 8.04 (m, 2H), 6.98 (dd, 1H), 2.98-3.12 (m, 9H), 2.37 (s, 3H).

Description 28

Methyl 4-(2-methyl-4-nitrophenyl)piperazine-1-carboxylate (D28)

A stirred solution of D27 (1.00 g, 4.5 mmole) and diisopropylethylamine (0.77 g, 6.0 mmole) in DCM (20 ml) was treated dropwise with methyl chloroformate (0.51 g, 5.4 mmole). After 4 h the mixture was washed with diluted K₂CO₃ solution, dried (Na₂SO₄) and evaporated in vacuo to afford the title compond as a yellow-orange solid (1.09 g, 86%). MH⁺ 280.

¹H NMR (250 MHz, CDCl₃) δ 8.05 (m, 2H), 7.05 (dd, 1H), 3.43 (m, 4H), 3.12 (m, 4H), 2.87 (s, 3H), 2.38 (s, 3H).

Description 29

Methyl 4-(4-amino-2-methylphenyl)piperazine-1-carboxylate (D29)

The title compound was prepared from D28 (1.05 g, 3.99 mmole) using a similar method to Desription 26 as a buff powder (0.73 g, 72%).

¹H NMR (250 MHz, CDCl₃) δ 6.81 (d, 1H), 6.48-6.57 (m, 2H), 3.72 (s, 3H), 3.56 (m, 6H), 2.76 (m, 4H), 2.23 (s, 3H).

### EXAMPLE 1

cis-N-[3-Chloro-2-fluorophenyl]-5-(3,4,5-trimethylpiperazin-1-yl)benzofuran-3-carboxamide (E1)

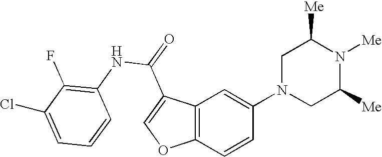

A stirred solution of 3-chloro-2-fluoroaniline (58 mg, 0.40 mmole) in toluene (4 ml) under argon was treated with trimethylaluminium (0.22 ml of 2M solution in toluene, 0.44 mmole) and maintained at room temp for 0.5 h, then a solution of D9 (50 mg, 0.16 mmole) in toluene (3 ml) was added and the mixture heated at 100° C. for 4 h. The solution was cooled, then added to 10% Na₂CO₃ solution (30 ml) and extracted with EtOAc (30 ml). The extract was dried (Na₂SO₄), concentrated under vacuum and the residue chromatographed on silica gel eluting with 04% MeOH/DCM to afford the title compound as a pale yellow oil (40 mg, 60%). This was converted to its HCl salt as a white solid.

¹H NMR (250 MHz, CDCl₃) δ 8.44-8.35 (m, 1H), 8.18 (s, 1H), 7.88 (br s, 1H), 7.50-7.44 (m, 2H), 7.20-7.14 (m, 3H), 3.49 (br d, 2H), 2.65 (t, 2H), 2.53-2.40 (m, 2H), 2.35 (s, 3H), 1.20 (d, 6H). MH⁺ 416.

The following Examples were prepared by a similar procedure to that of E1 using the appropriate aniline and benzofuran ester (D9, D10, D15 or D16).

| Example | MH⁺ |
|---|---|
| cis-N-[2-Fluoro-3-(trifluoromethyl)phenyl]-5-(3,4,5- | 450 |
| trimethylpiperazin-1-yl)benzofuran-3-carboxamide (E2) | |
| cis-N-[2,3-Dichlorophenyl]-5-(3,4,5-trimethylpiperazin-1- | 432 |
| yl)benzofuran-3-carboxamide (E3) | |
| cis-N-[2′-Methyl-4′-(5-methyl-1,2,4-oxadiazol-3-yl)-4-biphenyl]- | 536 |
| 5-(3,4,5-trimethylpiperazin-1-yl)benzofuran-3-carboxamide | |
| (E4) from aniline D12 | |
| cis-N-[2′-Methyl-4′-(5-methyl-1,2,4-oxadiazol-3-yl)-4-biphenyl]- | 522 |
| 5-(3,5-dimethylpiperazin-1-yl)benzofuran-3-carboxamide (E5) | |
| from aniline D12 | |
| cis-N-[3-Chloro-2-fluorophenyl]-6-fluoro-5-(3,4,5- | 434 |
| trimethylpiperazin-1-yl)benzofuran-3-carboxamide (E6) | |
| cis-N-[3-Chloro-2-fluorophenyl]5-(3,5-dimethylpiperazin-1-yl)- | 420 |
| 6-fluorobenzofuran-3-carboxamide (E7) | |
| cis-N-[3-Methyl-4-(6-methylpyridin-2-yl)phenyl]-5-(3,4,5- | 469 |
| trimethylpiperazin-1-yl)benzofuran-3-carboxamide (E8) from | |
| aniline Description 18 in WO 99/31086 | |
| cis-N-[3-Methyl-4-(6-methylpyridin-2-yl)phenyl]-5-(3,4,5- | 487 |
| trimethylpiperazin-1-yl)-6-fluorobenzofuran-3-carboxamide | |
| (E9) from aniline Description 18 in WO 99/31086 | |
| cis-N-[4-(Imidazol-1-yl)-3-methylphenyl]-5-(3,4,5- | 444 |
| trimethylpiperazin-1-yl)benzofuran-3-carboxamide (E10) | |
| cis-N-[4-(Imidazol-1-yl)-3-methylphenyl]-6-fluoro-5-(3,4,5- | 462 |
| trimethylpiperazin-1-yl)benzofuran-3-carboxamide (E11) | |
| cis-N-[3-Chloro-4-(pyridin-4-yl)phenyl]-5-(3,4,5- | 476 |
| trimethylpiperazin-1-yl)benzofuran-3-carboxamide (E12) from | |
| aniline Description 4 in WO 9931086 | |
| cis-N-[3-Chloro-4-(pyridin-4-yl)phenyl]-6-fluoro-5-(3,4,5- | 494 |
| trimethylpiperazin-1-yl)benzofuran-3-carboxamide (E13) from | |
| aniline Description 4 in WO 9931086 | |
| cis-N-[4-(4-Methoxycarbonylpiperazin-1-yl)-3-methylphenyl]- | 520 |
| 5-(3,4,5-trimethylpiperazin-1-yl)benzofuran-3-carboxamide | |
| (E14) | |
| cis-N-[2-(4-Cyanophenyl)-3-methylpyridin-5-yl]-6-fluoro-5- | 498 |
| (3,4,5-trimethylpiperazin-1-yl)benzofuran-3-carboxamide | |
| (E15) | |

### EXAMPLE 16

cis-N-[4-(4-Methoxycarbonylpiperazin-1-yl)-3-methylphenyl]-5-(3,4,5-trimethylpiperazin-1-yl)benzothiophene-3-carboxamide (E16)

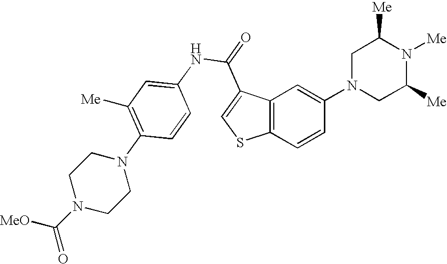

The title compound was prepared from D20 and D29 using a similar procedure to Example 1 as a buff-coloured solid (20%). MH⁺ 536.

¹H NMR (250 MHz, CDCl₃) δ 7.90 (m, 2H), 7.73 (d, 2H), 7.40 (m, 2H), 7.14 (dd, 1H), 7.00 (d, 1H), 3.73 (s, 3H), 3.56 (m, 6H), 2.82 (m, 4H), 2.59 (t, 2H), 2.34 (m, 8H), 1.20 (m, 6H).

The following Examples were prepared by a similar procedure to that of E1 using the appropriate aniline and benzothiophene ester (D20 or D21).

| Example | MH⁺ |
|---|---|
| cis-N-[2,3-Dichlorophenyl]-5-(3,4,5-trimethylpiperazin-1- | 448 |
| yl)benzothiophene-3-carboxamide (E17) | |
| cis-N-[4-(Imidazol-2-yl)-3-methylphenyl]-5-(3,4,5- | 478 |
| trimethylpiperazin-1-yl)benzothiophene-3-carboxamide (E18) | |
| cis-N-[Indol-5-yl]-5-(3,4,5-trimethylpiperazin-1- | 419 |
| yl)benzothiophene-3-carboxamide (E19) | |
| cis-N-[2′-Methyl-4′-(5-methyl-1,2,4-oxadiazol-3-yl)-4-biphenyl]- | 552 |
| 5-(3,4,5-trimethylpiperazin-1-yl)benzothiophene-3- | |
| carboxamide (E20) | |
| cis-N-[2,3-Dichlorophenyl]-5-(3,5-dimethylpiperazin-1- | 434 |
| yl)benzothiophene-3-carboxamide (E21) | |
| cis-N-[2′-Methyl-4′-(5-methyl-1,2,4-oxadiazol-3-yl)-4-biphenyl]- | 538 |
| 5-(3,5-dimethylpiperazin-1-yl)benzothiophene-3-carboxamide | |
| (E22) | |

### EXAMPLE 23

cis-3-[2′-Methyl-4′-(5-methyl-1,2,4-oxadiazol-3-yl)-1,1′-biphenyl-4-carbonyl]-5-(3,4,5-trimethylpiperazin-1-yl)-1H-indole (E23)

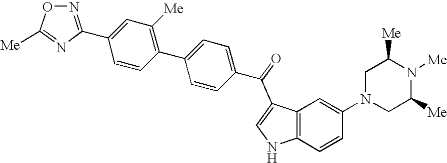

To a solution of D27 (500 mg, 2.05 mmole) in DCM (20 ml) at 10-15° C. under argon was added a solution of ethyl magnesium bromide (3.0M solution in Et₂O, 1.35 ml, 4.1 mmole) over 5 mins and the mixture stirred under argon for 30 mins whilst maintaining the temperature at 10-15° C. To the yellow precipitate was added a solution of 2′-methyl-4′-(5-methyl-1,2,4-oxadiazol-3-yl)biphenylcarbonyl chloride (prepared from the carboxylic acid ref: EP 0533268A1 by treatment with thionyl chloride) (680 mg, 2.17 mmole) in DCM (20 ml) and stirring continued for 2 h. The mixture was treated with 2M HCl (40 ml) and THF (50 ml) and stirred vigorously before basification with 10% aqueous Na₂CO₃ solution. The organic layer was separated, dried (Na₂SO₄) and concentrated under vacuum. Chromatography on silca gel eluting with 5-20% MeOH/DCM followed by preparative HPLC (Biotage FLEX) afforded the title compound as a yellow solid (36 mg, 3%). MH⁺ 520.

¹H NMR (250 MHz, CDCl₃) δ 8.68 (br s, 1H), 8.01 (s, 2H), 7.95 (d, 1H), 7.90 (d, 2H), 7.71 (d, 1H), 7.46 (d, 2H), 7.37 (m, 2H), 7.07 (dd, 1H), 3.53 (br d, 2H), 2.68 (s, 3H), 2.67 (m, 2H), 2.49 (m, 2H), 2.38 (s, 3H), 2.35 (s, 3H), 1.20 (d, 6H).

### EXAMPLE 24

cis-1-Methyl-3-[2′-methyl-4′-(5-methyl-1,2,4-oxadiazol-3-yl)-1,1′-biphenyl-4-carbonyl]-5-(3,4,5-trimethylpiperazin-1-yl)-1H-indole

To a solution of E23 (25 mg, 0.05 mmole) in THF (10 ml) was added sodium hydride (60% dispersion in oil, 2 mg, 0.05 mmole). The mixture was stirred at room temp for 30 mins. under argon before addition of methyl iodide (0.005 ml, 0.08 mmole), then stirring was continued for 2 h. The THF was removed in vacuo and the residue partitioned between DCM and 10% aqueous Na₂CO₃ solution. The organics were dried (Na₂SO₄) and evaporated to a yellow solid. Attempted preparative HPLC failed due to the compound's high insolubility, however the compound was found to crystallise from hot DMSO/MeOH. The crystals were washed well with water, followed by Et₂O, and dried in vacuo to afford the title compound as a yellow solid (5 mg, 20%). MH⁺ 534. HPLC >90% purity.

1. A compound of formula (I) or a pharmaceutically acceptable salt thereof: 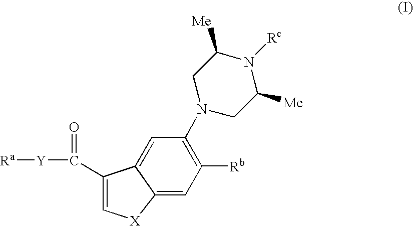
in which R^{a} is a group of formula (i) 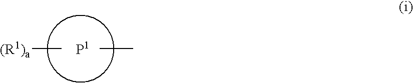
wherein P¹ is phenyl, naphthyl or heteroaryl;
R¹ is halogen, C₁₋₆alkyl, C₃₋₆cycloalkyl, COC₁₋₆alkyl, C₁₋₆alkoxy, hydroxy, hydroxyC₁₋₆alkyl, nitro, CF₃, cyano, SR⁶, SOR⁶, SO₂R⁶, SO₂NR⁶R⁷, CO₂R⁶, CONR⁶R⁷, OCONR⁶R⁷, NR⁶R⁷, NR⁶COR⁷, NR⁶CO₂R⁷, NR⁶SO₂R⁷, NR⁶CONR⁷R⁸, CH₂NR⁶COR⁷, CH₂NR⁶CO₂R⁷, CH₂NR⁶SO₂R⁷, CR⁶═NOR⁷ where R⁶, R⁷ and R⁸ are independently hydrogen or C₁₋₆alkyl,
a is 0, 1, 2 or 3;
or R^{a} is a group of formula (ii) 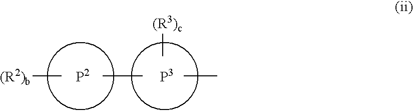
wherein
P² is phenyl, naphthyl, heteroaryl or a 5 to 7 membered heterocyclic ring;
P³ is phenyl, naphthyl or heteroaryl;
R² is as defined above for R¹ in formula (i) or R² is heteroaryl optionally substituted by C₁₋₆alkyl, halogen or COC₁₋₆alkyl or is a 5-7 membered heterocyclic ring optionally substituted by oxo;
R³ is halogen, C₁₋₆alkyl, C₃₋₆cycloalkyl, C₁₋₆alkoxy, COC₁₋₆alkyl, hydroxy, nitro, CF₃, cyano, CO₂R⁶, CONR⁶R⁷, NR⁶R⁷ where R⁶ and R⁷ are as defined above;
b and c are independently 0, 1, 2 or 3;
Y is a single bond, CH₂ or NH;
X is O, S or N—R⁵ where R⁵ is hydrogen or C₁₋₆alkyl;
R^{b} is hydrogen, halogen, C₁₋₆alkyl, CF₃, COC₁₋₆alkyl or cyano;
R^{c} is hydrogen or C₁₋₆alkyl.

2. A compound according to claim 1 in which R^{a} is a group of formula (i) wherein P¹ is phenyl.

3. A compound according to claim 1 in which R^{a} is a group of formula (ii) wherein P² and P³ are independently phenyl or pyridyl.

4. A compound according to any of the preceding claims in which R^{c} is hydrogen or methyl.

5. A compound according to claim 1 which is a compound E1-E24 (as described above) or a pharmaceutically acceptable salt thereof.

6. A process for the preparation of a compound of formula (I) according to claim 1 or a pharmaceutically acceptable salt thereof which comprises:
(a) when Y is NH, coupling a compound of formula (II):
R^{a}—NH₂ (II)
in which R^{a} is as defined in formula (I) with a compound of formula (III): 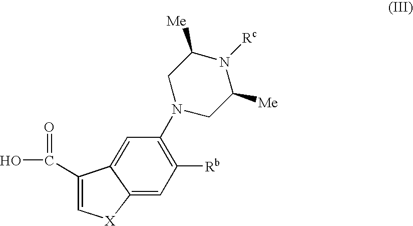
in which X, R^{b} and R^{c} are as defined in formula (I); or
(b) when Y is NH, coupling a compound of formula (II):
R^{a}—NH₂ (II)
in which R^{a} is as defined in formula (I) with a compound of formula (IV): 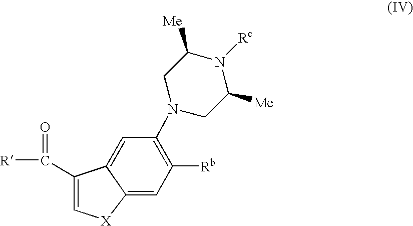
in which X, R^{b} and R^{c} are as defined in formula (I) and R′ is a C₁₋₆alkoxy group; or
(c) when Y=a single bond or CH₂, reacting a compound of formula (V); 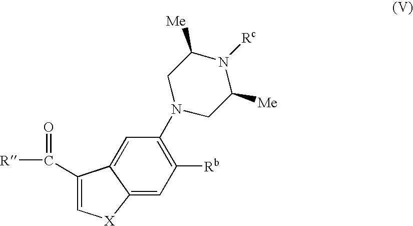
in which X, R^{b} and R^{c} are as defined in formula (I) and R″ is hydrogen, chloro, di-C₁₋₆alkylamino or C₁₋₆alkylC₁₋₆alkoxyamino with a compound of formula (VI)
R^{a}—(CH₂)_{q}-M (VI)
in which R^{a} is as defined for formula (I), q is 0 or 1 and M is Mg, Zn, Cd or Li; or
(d) reacting a compound of formula (VII) 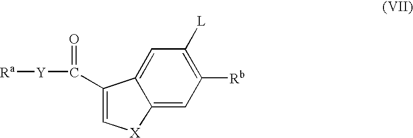
in which R^{a}, X, Y and R^{b} are as defined in formula (I) and L is a bromine or iodine with a compound of formula (VIII) 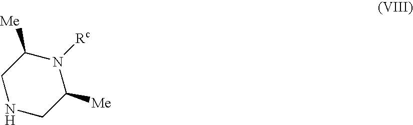
in which R^{c} is as defined in formula (I); or
(e) when X=NH and Y=a single bond or CH₂, reacting a compound of formula (IX): 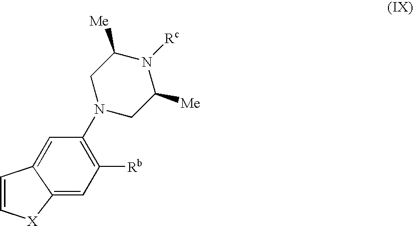
in which R^{b} and R^{c} are as defined in formula (I), with a compound of formula (X)
R^{a}—(CH₂)_{q}—COCl (X)
in which R^{a} is as defined for formula (I) and q is 0 or 1;
and optionally thereafter for either process (a), (b), (c), (d) or (e):
removing any protecting groups, and/or
converting a compound of formula (I) into another compound of formula (I), and/or
forming a pharmaceutically acceptable salt.

7. A compound according to any one of claims 1 to 5 for use in therapy.

8. A compound according to any one of claims 1 to 5 for use in the treatment of depression.

9. A pharmaceutical composition which comprises a compound according to any of claims 1 to 5 or a pharmaceutically acceptable salt thereof and a pharmaceutically acceptable carrier or excipient.

10. A compound of formula (I) as defined in any one of claims 1 to 5 or a pharmaceutically acceptable salt thereof, for use in the treatment of diseasea where an antagonist of the 5-HT_{1B} receptor is beneficial.

11. A method of treating a disease where an antagonist of the 5-HT_{1B} receptor is beneficial, which comprises administering a safe and therapeutically effective amount of compound of formula (I) or a pharmaceutically acceptable salt to a patient in need thereof.

12. A method as claimed in claim 11, wherein the disease is depression.

13. The use of a compound of formula (I) as defined in any one of claims 1 to 5 or a pharmaceutically acceptable salt thereof, in the manufacture of a medicament for the treatment of a disease where an antagonist of the 5-HT_{1B} receptor is beneficial.

14. The use as claimed in claim 13, wherein the disease is depression.
